(19) United States
(12) Patent Application Publication
(10) Pub. No.: US 2009/0248163 A1
(43) Pub. Date: Oct. 1, 2009

(54) SPINAL SURGERY INTERBODY

(76) Inventors: Emily E. King, Georgetown, TX (US); Wayne P. Gray, JR., Pflugerville, TX (US); Ryan C. Harvey, Bordeaux (FR)

Correspondence Address: SPRINKLE IP LAW GROUP, 1301 W. 25TH STREET, SUITE 408, AUSTIN, TX, 78705, US

(21) Appl. No.: 12/059,982

(22) Filed: Mar. 31, 2008

(51) Int. Cl. A61F 2/44; A61B 17/56

(52) U.S. Cl. 623/17.16; 606/86 A

(57) ABSTRACT

An inter-vertebral implant wherein at least one of the side walls defining a hole from the exterior of the implant to the fusion promotion cavity and having a posterior portion and an anterior portion, the side wall hole being elongated in a direction between the posterior and anterior ends of the side wall hole. In some embodiments, the side wall hole is tapered toward the posterior portion of the implant and can be tear drop shaped. In some embodiments the cephalad and caudal surfaces may be convex with radii of curvature which can be the same for all implant sizes. The surfaces may include teeth which can extend beyond a central cavity. A nose, tapered in the saggital and transverse planes may be included on the anterior end of the implant. Some embodiments also include a threaded hole (with a depth that can be the same for all implant sizes) and an adjoining slot for engaging an insertion instrument. Each of the portions of the implant can have the same length for all implant sizes.

### TECHNICAL FIELD OF THE DISCLOSURE

Embodiments of the disclosure relate generally to implants for spinal surgery and more particularly to artificial interbody spinal fusion implants for insertion within implantation spaces formed across the height of disc spaces between vertebral bodies of human spines. Embodiments of the disclosure relate to interbody spinal implants that may be inserted into a patient during surgical procedures including minimally invasive surgical procedures.

### BACKGROUND OF THE DISCLOSURE

Bone may be subject to degeneration caused by trauma, disease, and/or aging. Degeneration may destabilize the affected bone and affect surrounding structures. For example, destabilization of a spine may result in an alteration of the natural spacing between adjacent vertebral bodies. Alteration of the natural spacing between adjacent vertebral bodies may subject the nerves that pass between the vertebral bodies to pressure. Pressure applied to the nerves may cause pain and/or nerve damage. Maintaining the natural spacing between vertebral bodies may reduce the pressure applied to these nerves. A spinal interbody implant may be used to maintain or restore the natural spacing between vertebral bodies.

Spinal implants may be inserted during a vertebral bodies replacement or interbody fusion procedure using a posterior approach as in Posterior Lumbar Interbody Fusion (PLIF) or Transforaminal Lumbar Interbody Fusion (TLIF). Spinal implants may be inserted during a spinal stabilization procedure using an anterior spinal approach as in Anterior Lumbar Interbody Fusion (ALIF). With these and other procedures, spinal implants may also be inserted into implantation spaces between the C2 and S1 vertebral bodies.

Conventional procedures for inserting spinal implants require distracting the adjacent vertebral bodies with a distractor or series of distractors then inserting the spinal implant in the distracted space. These procedures require a number of complex surgical steps and tools that can make the procedures time consuming and complicated.

### SUMMARY OF THE DISCLOSURE

Embodiments of the present disclosure provide spinal interbody implants that eliminate, or at least substantially reduce, the shortcomings of prior art spinal implants.

One embodiment provides an interbody implant indicated for vertebral body replacement or interbody fusion. The implant may be inserted through an open, or minimally invasive, posterior, anterior, or transforaminal approach into the implantation space to maintain or restore the height of a disc space after a discectomy or other procedure. Fusion of the vertebral bodies may take place over the course of 6-12 months during which it may be desired to maintain an appropriate space between the vertebral bodies. In some embodiments, the implants come in a variety of sizes which may be selected from to match patient anatomy.

One embodiment provides an artificial interbody spinal fusion implant for insertion within an implantation space formed across the height of a disc space between vertebral bodies of a human spine. The implant can comprise a posterior portion, an anterior portion opposite from the posterior portion and a pair of medial-lateral sidewalls between the posterior portion and the anterior portion. The posterior portion, anterior portion, and side walls can define an implant body having a cephalad surface, a caudal surface, and a fusion promotion cavity extending between the cephalad surface and the caudal surface. According to various embodiments, one or both of the cephalad and caudal surfaces can be convex. At least one of the sidewalls defines a hole from the exterior of the implant to the fusion promotion cavity. The hole can be centered about a primary axis of impact and can be tapered toward the posterior portion of the implant along the axis of impact. For example, the side wall hole can be tear drop shaped or have another tapered shape that is thinner towards the posterior portion of the implant.

Various embodiments of implants can include features to facilitate surgical procedures. For example, according to one embodiment, the anterior portion of the implant can be tapered in the saggital and transverse planes. As another example, one or more of the caudal or cephalad surfaces can include teeth that project along the caudal and cephalad surfaces beyond the fusion promotion cavity. According to further embodiments, the posterior portion of the implant can define a threaded instrument engagement hole for engagement with an instrument for inserting the implant. The posterior portion can further include an engagement notch adjoining the instrument engagement hole. Embodiments of implants can also include fluoroscopy markers. According to various embodiments, a first and second elongated fluoroscopy marker can be located toward the posterior portion of the implant, while a third fluoroscopy marker can be located toward the anterior portion of the implant. According to an embodiment, the third fluoroscopy marker has approximately the same cross sectional area as the first fluoroscopy marker in a first viewing plane and has a different cross sectional area than the first fluoroscopy marker in a second viewing plane orthogonal to the first viewing plane.

Other embodiments can include a surgical kit for surgery on human spines having vertebral bodies and disc spaces having heights between some of the vertebral bodies, the vertebral bodies having an anterior aspect and a posterior aspect and a depth therebetween. According to one embodiment, a surgical kit can comprise a set of different sized artificial interbody spinal fusion implants for insertion within implantation spaces formed across the heights of the disc spaces. Each implant can further comprise a posterior portion of the implant shaped to interface with an insertion tool, an anterior portion of the implant opposite the posterior portion of the implant and a pair of medial-lateral side walls of the implant between the posterior and anterior portions of the implant. The posterior portion, anterior portion, and medial lateral sidewalls can define an implant body having a cephalad surface, a caudal surface and defining a fusion promotion cavity extending between the cephalad surface and the caudal surface. According to an embodiment, at least one of the side walls defines a hole from the exterior of the implant to the fusion promotion cavity, the side wall hole being centered about a primary axis of impact and being tapered toward the posterior portion of the implant along the axis of impact. For example, the side wall hole can be tear drop shaped or have another tapered shape that is thinner towards the posterior portion of the implant.

According to an embodiment, a posterior portion of each implant in a kit defines a threaded instrument engagement hole for engagement with the instrument and an instrument engagement notch adjoining the instrument engagement hole. The instrument engagement hole of each implant has a depth in a direction along the axis of impact, the depth being the same for all sizes in some embodiments.

The cephalad and caudal surfaces of each implant optionally can be convex with each of the cephalad surfaces having a first radius of curvature and each of the caudal surfaces having a second radius of curvature. The first radius of curvature can be the same for all sizes and the second radius of curvature can be the same for all sizes.

According to an embodiment, each implant in the kit can have a posterior portion with a first length from a posterior portion to the fusion promotion cavity. The first length being the same for all sizes of implants in the kit. Similarly, according to and embodiment, each implant can have an anterior portion with a second length from an anterior portion to the fusion promotion cavity. The second length can be same for all sizes.

Each implant can further comprise a first and a second elongated fluoroscopy marker toward a posterior portion of the implant and a third fluoroscopy marker toward an anterior portion of the implant. At least one of the fluoroscopy markers can be a selected distance from one of the ends of the implant. This distance can be the same for all sizes.

Another embodiment can include a method of manufacturing an artificial interbody spinal fusion implant for insertion within an implantation space formed across the height of a disc space between vertebral bodies of a human spine, the vertebral bodies having an anterior aspect and a posterior aspect and a depth therebetween. The method can comprise forming a posterior portion of the implant shaped to interface with an insertion tool, forming an anterior portion of the implant opposite the posterior portion of the implant, and forming a pair of medial-lateral side walls of the implant between the posterior and anterior portions of the implant. The posterior portion, anterior portion, and medial lateral sidewalls can define an implant body having a cephalad surface, a caudal surface and defining a fusion promotion cavity extending between the cephalad surface and the caudal surface. The method can further comprise forming a hole in at least one of the medial-lateral side walls from the exterior of the implant to the fusion promotion cavity, the side wall hole being centered about a primary axis of impact and being tapered toward the posterior portion of the implant along the axis of impact. For example, the side wall hole can be a tear drop shape or other tapered shape. The method can further include forming a set of teeth into at least one of the cephalad or caudal surfaces. According to one embodiment, the teeth can be formed to extend beyond the cavity toward at least one of the ends of the implant.

According to various embodiments, at least one of the surfaces is convex and has a radius of curvature. The teeth can be formed such that the height of the teeth does not extend beyond the radius of curvature.

Embodiments of the method can further comprise forming at least a first and second marker hole in the implant toward the posterior portion of the implant and a third marker hole toward the anterior portion of the implant and inserting a first marker in the first fluoroscopy marker in the first marker hole, a second fluoroscopy marker in the second marker hole and a third fluoroscopy marker in the third marker hole. According to various embodiments, the third fluoroscopy marker has approximately the same cross sectional area as the first fluoroscopy marker in a first viewing plane and has a different cross sectional area than the first fluoroscopy marker in a second viewing plane orthogonal to the first viewing plane.

Embodiments of the present described herein provide advantages over previous implants. One advantage provided by various embodiments is that the implant can be shaped to better distribute the stress caused by impacts to the implant during the insertion process. Another advantage provided by various embodiments of the implant is that the nose can aid in distraction and can be shaped to move aside nerves during insertion. Yet another advantage provided by embodiments of an implant is that a set of teeth can be provided to reduce the likelihood that the implant can be ejected from the implantation site. Another advantage provided by embodiments disclosed herein is that features of the implant can be more easily manufactured.

These, and other, aspects will be better appreciated and understood when considered in conjunction with the following description and the accompanying drawings. The following description, while indicating various embodiments and numerous specific details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE FIGURES

A more complete understanding of the disclosure and the advantages thereof may be acquired by referring to the following description, taken in conjunction with the accompanying drawings in which like reference numbers generally indicate like features and wherein:

FIG. 1 is a perspective view of an implant being inserted into an implantation space in accordance with some embodiments.

FIG. 2 illustrates an implant and insertion tool in accordance with some embodiments.

FIG. 3 is a perspective view of an implant in accordance with some embodiments.

FIG. 4 illustrates implants of different sizes in accordance with some embodiments.

FIG. 5 is a side elevation view of an implant in accordance with some embodiments.

FIG. 6 is a cross sectional view of an implant in accordance with some embodiments.

FIG. 7 is a perspective view of an implant in accordance with some embodiments.

FIG. 8 is a cross sectional view of an implant in accordance with some embodiments.

FIG. 9 is a top plan view of an implant in accordance with some embodiments.

FIG. 10 is a side elevation view of an implant in accordance with some embodiments.

### DETAILED DESCRIPTION

Preferred embodiments of the disclosure are illustrated in the FIGURES, like numerals being used to refer to like and corresponding parts of the various drawings. Embodiments of the disclosure provide artificial interbody spinal fusion implants for insertion within implantation spaces formed across the height of a disc spaces between vertebral bodies of human spines.

One embodiment of an implant can comprise a posterior portion, an anterior portion opposite from the anterior portion and a pair of medial-lateral side walls between the posterior portion and the anterior portion. The posterior portion, anterior portion, and side walls can define an implant body having a cephalad surface, a caudal surface and defining a fusion promotion cavity extending between the cephalad surface and the caudal surface. According to various embodiments, one or more of the cephalad and caudal surfaces can be convex. At least one of the sidewalls defines a hole from the exterior of the implant to the fusion promotion cavity. The hole can be centered about a primary axis of impact and can be tapered toward the posterior portion of the implant along the axis of impact. For example, the side wall hole can be tear drop shaped or have another tapered shape that is thinner towards the posterior portion of the implant.

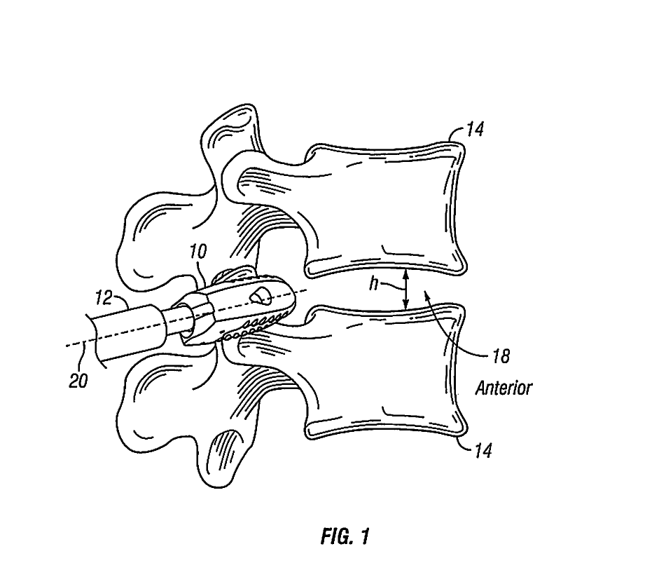

FIG. 1 is a diagrammatic representation of an insert 10 being inserted into a patient's spine with instrument 12. Implant 10 can be used to maintain, or restore, the height h between adjacent vertebral bodies 14 of the patient by being inserted in to implantation space 18. One or more implants 10 may be inserted after a discectomy in which the disc, which previously separated vertebral bodies 14, is removed. Implant 10 may be inserted during a vertebral body replacement or interbody fusion procedure using posterior or anterior approaches. Various techniques can be used to insert implant 10 into implantation space 18 such as Posterior Lumbar Interbody Fusion (PLIF), Transforaminal Lumbar Interbody Fusion (TLIF), or Anterior Lumbar Interbody Fusion (ALIF). With these and other procedures, spinal implants may also be inserted into implantation spaces between the C2 and S1 vertebral bodies.

Implant 10 can include a cavity into which bone graft material may be placed to promote fusion of vertebral bodies and may include a hole(s) in one or both of its sidewalls to allow bone cells to migrate into the fusion promotion cavity. The hole can be circular, elongated, tear drop shaped or have some other shape. Implant 10 may define cephalad and caudal surfaces which may be convex to correspond to the anatomical surfaces (of vertebral bodies 14) with which the surfaces abut when implant 10 is in implantation space 18. One or both surfaces may include teeth to prevent expulsion of implant 10 from implantation space 18. In some embodiments, implant 10 may define a threaded posterior hole and adjacent notch or other features to engage corresponding features of instrument 12 to detachably attach and lock implant 10 to instrument 12. FIG. 1 also illustrates that implant 10 may be inserted into implantation space 18 along axis of impact 20. Insertion may occur via striking the proximal end of instrument 12 with a slap hammer, mallet, etc. In some embodiments, implant 10 may include an anterior nose portion tapered in the saggital and transverse planes to allow implant 10 to be self-distracting when being inserted between vertebral bodies 14. Medial-lateral edges and the corners of implant 10 may be rounded to aid in implant's 10 self-distraction. Markers with the same or different configurations may be provided in implant 10 to enable surgical personnel (via a fluoroscope or other instrument) to identify the position and orientation of implant 10 when implant 10 is in a patient's body. Any number of markers may be used to facilitate placement of implant 10.

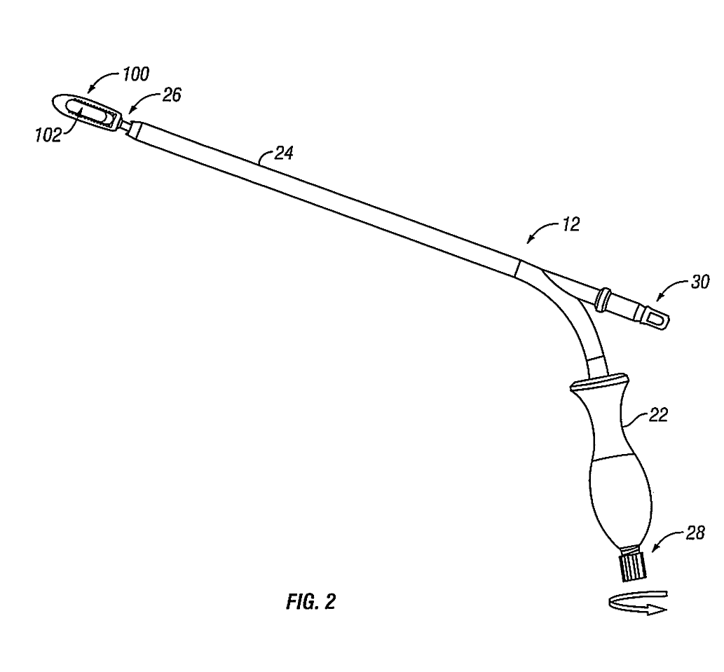

FIG. 2 is a diagrammatic representation of one embodiment of an instrument 12 for inserting an implant. Instrument 12, in FIG. 2, is illustrated as engaging the posterior portion of implant 10. As illustrated by FIG. 2 instrument 12 can include handle 22, shaft 24, features 26 for engaging implant 10, actuator 28, and attachment point 30 for an impaction cap, an extension, or other accessory. Features 26 may engage corresponding features on the posterior portion of implant 10 to detachably attach implant 10 to instrument 12. According to some embodiments, features 26 can include a threaded member to connect to threads of implant 10 and lock implant 10 to features 26 of instrument 12 against rotation relative to instrument 12. In some embodiments, features 26 include a threaded member which can be connected to actuator 28 by a linkage such as a shaft internal to the tool 26. Rotation of actuator 28 can cause rotation of the threaded member. While the example of a threaded member is used, instrument 12 can include other features to engage with implant 10 such as, but not limited to, features that form an interference or frictional fit with implant 10, tabs that fit in corresponding slots of implant 10, detents, indents, or other features that removably couple implant 10 to instrument 12.

Instrument 12 can include a handle 22 that can be designed for an ergonomic grip. Handle 22 can be detachable from or fixed to other portions of instrument 12 and can be positioned so that the surgeon's hand is out of the line of sight to implant 10. Additionally, handle 22 can be positioned to be out of the way of a slap hammer, mallet, or other device used to drive implant 10 into the body. In this regard, instrument 12 can include a feature to allow an impact to be applied. According to some embodiments, instrument 12 can include an impaction cap for a slap hammer. The impaction cap may be permanently affixed to instrument 1 or may be removably coupled to instrument 12 by threads or other mechanisms. In the example illustrated in FIG. 2, instrument 12 includes a quick connect 30 attachment point to allow attachment of the impaction cap.

Handle 22 allows surgical personnel to navigate implant 10 through an incision in the patient's body and to the surgical site near vertebral bodies 14. During a procedure, instrument 12 can be used to place the anterior portion of implant 10 between adjacent vertebral bodies 14 (as shown in FIG. 1) but just outside of implantation space 18 (as shown in FIG. 1) thereby positioning implant 10 for insertion therebetween. With an impaction cap on attachment point 30, surgical personnel can impact instrument 12 with a mallet or other device to drive implant 10 in to implantation space 18 (as shown in FIG. 1). Actuator 28 can be used to unlock and release implant 10 from instrument 12. Instrument 12 can be removed from the patient's body using handle 22.

In some embodiments, surgical personnel may, instead of unlocking and releasing implant 10, may withdraw implant 10 from implantation space 18 using instrument 12. Surgical personnel can remove implant 10 from instrument 12 and replace it with another implant (perhaps with differing overall dimensions). Surgical personnel can insert replacement implant 10 into implantation space 18. Originally attached implant 10 and replacement implant 10, as well as instrument 12, the mallet, and any extensions or accessories for instrument 12 may be included in a kit.

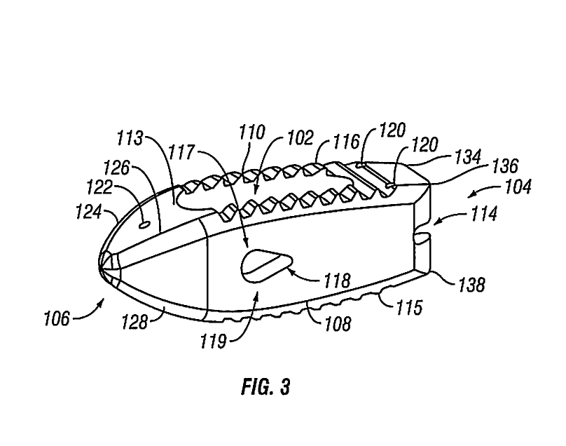

With reference now to FIG. 3, FIG. 3 illustrates a perspective view of implant 100 constructed in accordance with some embodiments. Implant 100 can correspond to implant 10 of FIG. 1. FIG. 3 illustrates various features of implant 100 according to some embodiments. FIG. 3 illustrates cavity 102, posterior portion 104, anterior portion 106, medial-lateral side wall 108, and medial-lateral side wall 110. Sidewall hole 118 and regions 117 and 119 are also illustrated. Medial-lateral edges 124, 126, and 128 and corners 134, 136, and 138 also appear in FIG. 3. Cephalad surface 113 and teeth 116 are also illustrated by FIG. 3. Fluoroscopy markers 120 and 122 and instrument engagement notch 114 also appear in FIG. 3.

Implant 100 can be formed of a biocompatible material such as PEEK, titanium, a titanium-aluminum alloy such as Ti6A14-Eli. Implant 100 may define cavity 102 wherein boney material may be placed to promote bone fusion between vertebral bodies 14. In various embodiments, implant 100 includes posterior portion 104, anterior portion 106, and two medial-lateral side walls 108 and 110. Cavity 102 may be generally centered in implant 100 between posterior portion 104, anterior portion 106, and medial-lateral side walls 108 and 110. In some embodiments, cavity 102 is elongated in a direction between posterior and anterior portions 104 and 106. The bone graft material which can be placed in cavity 102 can be natural or synthetic and can include by way of example, but not limitation, autograft bone such as bone from the patient's liliac crest, autograft bone from other locations, synthetic bone or a combination thereof. Furthermore, when the bone graft material is natural it can be gathered from the patient, a donor, or perhaps an animal source such as is the case with bovine bone graft material. In addition to the bone graft material, various carrier materials to retain the bone graft material in cavity 102 can be placed in cavity 102. Other materials, such as bone growth promotion materials, may be included in cavity 102.

With reference again to FIG. 3, implant 100 may define cephalad and caudal surfaces 113 and 115 respectively. Cephalad and caudal surfaces 113 and 115 are the portions of implant 100 closest to the exposed ends of vertebral bodies 14 (of FIG. 1) when implant 100 is in implantation space 18. Since a gap may exist between vertebral bodies 18 and implant 100 in at least some places, cephalad and caudal surfaces 113 and 115 can be shaped such that the gap is minimized to facilitate fusion of vertebral bodies 14. In some embodiments, one or both of cephalad and caudal surfaces 113 and 115 can be convex such that they correspond to the concave ends of vertebral bodies 14. In various embodiments, one or both of cephalad and caudal surfaces 113 can be concave, flat, or any other shape suitable for abutting vertebral bodies 14 particularly the anatomical domes of the corresponding vertebrae. Caudal surface 115 and cephalad surface 113 can be coated with titanium plasma spray, bone morphogenic proteins, hydroxyapatite, or other coatings to promote osseointegration. Outer surfaces of implant 100 can be roughed by processes such as, but not limited to, chemical etching, surface abrading, shot peening, electric discharge roughening, or embedding particles.

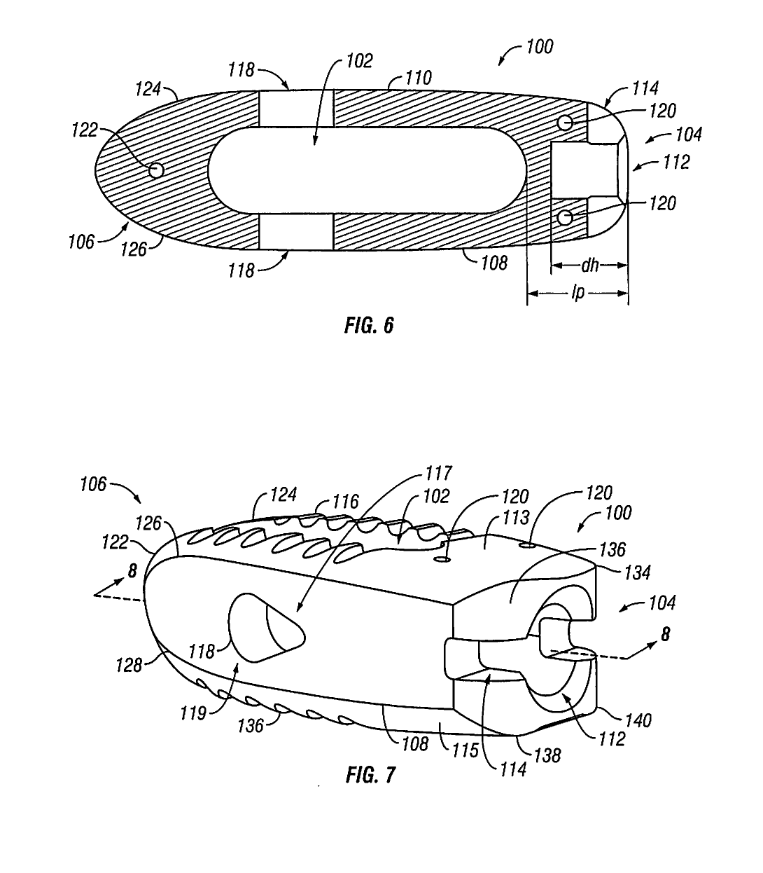

As described with respect to FIG. 2, to insert implant 100 into implantation space 18, surgical personnel can impact instrument 12 with a mallet to drive implant 100 into place. For an implant to be inserted into implantation space 18, though, often requires that adjacent vertebral bodies 14 be distracted. However, anterior portion 106 can be tapered in a posterior-anterior direction so that implant 100 may be self-distracting when inserted between vertebral bodies 14. Tapering of anterior portion 106 can be in the saggital and transverse planes as illustrated in FIG. 3. Lateral-medial edges 124, 126, and 128 of anterior end 106 (and of medial-lateral side walls 108 and 110) can be rounded to facilitate insertion of implant 100 into the implantation space 18. In various embodiments, posterior corners 134, 136, and 138 can be rounded (See FIG. 6).

With continuing reference to FIG. 3, once implant 100 is inserted into implantation space 18 (of FIG. 1), it is sometimes desired to determine the location and orientation of implant 100. To do so, surgical personnel can use a fluoroscope to locate implant 100 within the patient's body. Since fluoroscopes detect certain materials better than other materials, fluoroscopy markers 120 and 122 can be provided as part of implant 100 in some embodiments. Fluoroscopy markers 120 and 122 can be made of tantalum, titanium, stainless steel, etc. (or some other material detectable by fluoroscopy or other surgical visualization tools) and implant 100 can be made of polyetheretherketone (PEEK) or some other biologically compatible polymer, ceramic or other material. Fluoroscopy markers 120 and 122 may be shaped, sized, positioned, and oriented to allow surgical personnel or others to determine the position and orientation of implant 100 when it is in the patient's body. As FIG. 3 shows, fluoroscopy markers 120 and 122 can be positioned in implant 100 with fluoroscopy makers 120 positioned toward posterior portion 104 and fluoroscopy marker 122 positioned toward anterior portion 106. In some embodiments, fluoroscopy markers 120 can be similar in shape and size with fluoroscopy marker 122 being a different shape and size although fluoroscopy markers 120 and 122 can be similar in size, shape, orientation, etc.

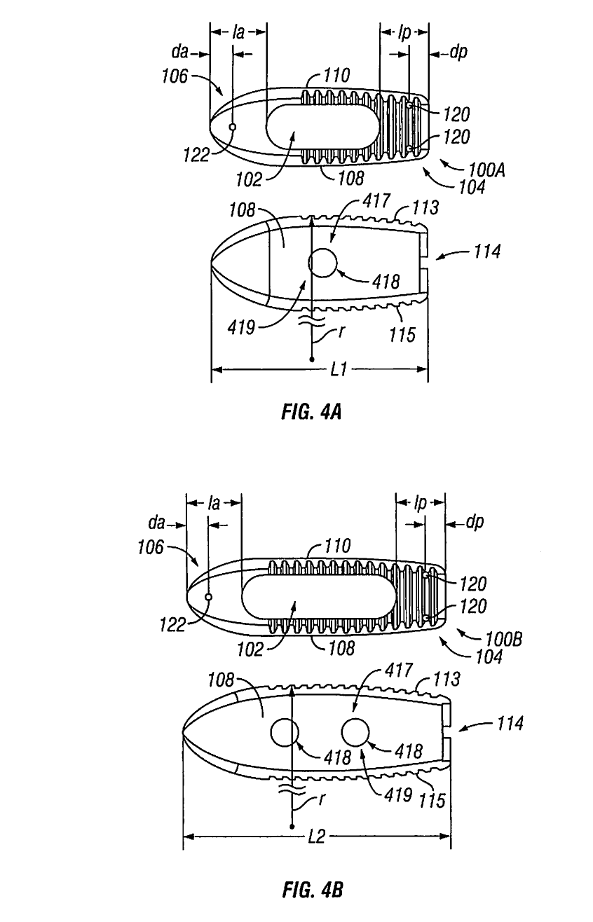

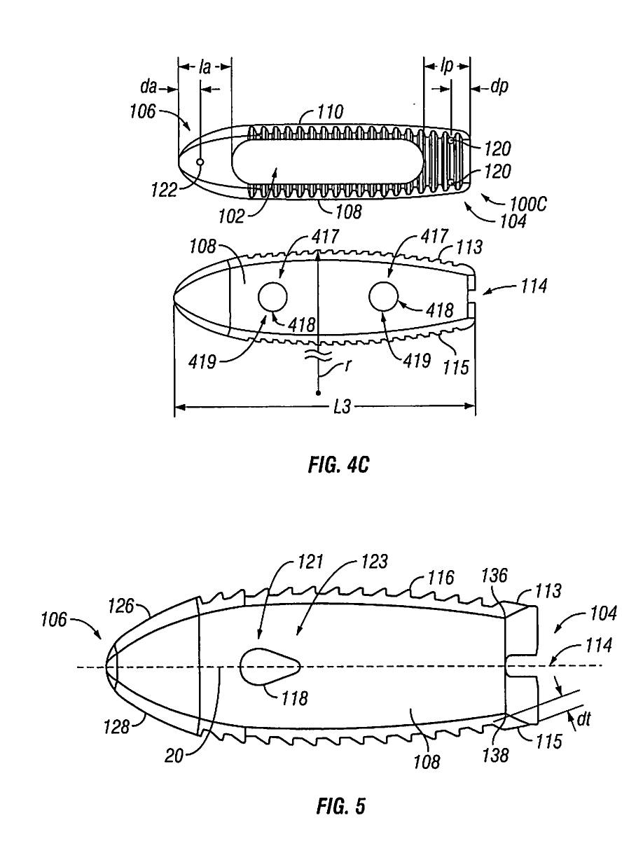

FIG. 4 illustrates various features of implant 100 according to some embodiments. FIG. 4 illustrates cavity 102, posterior portion 104, anterior portion 106, medial-lateral side wall 108, and medial-lateral side wall 110. Side wall hole 418 and regions 417 and 419 are also illustrated. As shown in the embodiment of FIG. 4, the sidewall holes can have a non-tapered shape. Fluoroscopy markers 120 and 122 also appear in FIG. 4. Instrument engagement notch 114 is illustrated in FIG. 4. In addition, FIG. 4 illustrates lengths I1-I3, Ip, and Ia and radius of curvature r.

As illustrated by FIG. 4, implant 100 can be nominally 25 mm, 30 mm, or 35 mm in length I1, I2, and I3 respectively. In some embodiments, overall length L of implant 100 can be a nominal 20 mm although other lengths L are within the scope of the disclosure. Table 1, below, lists the overall nominal dimensions (heights, lengths, and widths) of implants constructed in accordance with various embodiments.

TABLE 1

| | Height (mm) | | | | |
|---|---|---|---|---|---|
| Length (mm) | 7 | 9 | 11 | 13 | 15 |
| 25 | 9 & 11 | 9 & 11 | 9 & 11 | 9 & 11 | 9 & 11 |
| 30 | 9 & 11 | 9 & 11 | 9 & 11 | 9 & 11 | 9 & 11 |
| 35 | | 9 & 11 | 9 & 11 | 9 & 11 | 9 & 11 |

Those skilled in the art will recognize that many other implants of different overall dimensions are possible and within the scope of the disclosure.

In some embodiments, cephalad and caudal surfaces 113 and 115 have radius of curvature r (see FIG. 4) defining their respective degrees of curvature. Radius of curvature r can vary between implants 100, implant sizes, and cephalad and caudal surfaces 113 and 115 or it can be the same for all implants 100 and cephalad and caudal surfaces 113 and 115. In some embodiments, radius of curvature r is about 3″ for all implants 100 and both cephalad and caudal surfaces 113 and 115. In some embodiments, cephalad and caudal surfaces 113 and 115 can have circular profiles although the radius of curvature need not be constant across the length of implant 100.

In some embodiments, lengths Ip and Ia of posterior and anterior portions 104 and 106 respectively may vary between implants 100 and between implant sizes as illustrated by FIG. 4. In some embodiments, however, lengths Ip and Ia remain the same between implants 100 and implant sizes. Fixed lengths Ip and Ia can simplify the manufacture of implants 100 and reduce costs accordingly. In some embodiments, though, lengths Ip (length from posterior end to most posterior portion of cavity 102) and Ia (length from anterior end to most anterior portion of cavity 102) can be fixed at certain lengths such as about 0.22″ and about 0.25″.

Since patient anatomy varies widely, some embodiments provide a kit of implants 100 of differing heights, widths, and lengths including, but not limited to, those dimensions shown by FIG. 4 and Table 1. In some embodiments, combinations of the overall dimensions of implants 100 can be varied in increments to provide a wide variety of implants for selection for insertion into implantation space 18. Various sizes of implants 10 can be indicated by color, numbers, codes, packaging, or other indicators. In some embodiments, one or more of the overall dimensions can be varied by 1 mm or by a tenth or twentieth of an inch. Kits of various embodiments can include, besides implants 100, one or more insertion instruments 12 of FIG. 2. Some kits contain extensions to instrument 12, mallet(s) for impacting instrument 12 to drive implant 100 in to implantation space 18 (see FIG. 1), and other various accessories.

FIG. 5 illustrates a side view of implant 100 according to some embodiments. FIG. 5 illustrates posterior portion 104, anterior portion 106, and medial-lateral side wall 108. Side wall hole 118 is also illustrated. Medial-lateral edges 126 and 128 and corners 136 and 138 also appear in FIG. 5. FIG. 5 also illustrates teeth 116 formed in one, or both, of caudal and cephalad surfaces 113 and 115. Instrument engagement notch 114 is also illustrated in FIG. 5.

As mentioned previously, forming teeth 116 in the body of implant 100 with a depth of dt (See FIG. 5) allows more accurate matching of implant 100 height to implantation space height h. In some embodiments, tooth depth dt is about 0.02″. In some embodiments tooth depth dt is constant across the length and width of implant 100 although in some embodiments tooth depth dt varies across implant 100. In some embodiments, cephalad and caudal surfaces 113 and 115 can include knurls, ridges, grooves, etc. instead of or in combination with teeth 116. Teeth 116 may be deburred (as may other surfaces of implant 100) in some embodiments.

FIG. 5 also illustrates that side wall hole 118 can be positioned such that (when viewed from the side) the center of side wall hole 118 aligns with axis of impact 20. Side wall holes 118 can be positioned off the axis of impact 20 in some embodiments. According to various embodiments, sidewall hole 118 is tapered towards the posterior end such that the cross section of region 121 is smaller than region 123. Wile shown as having a generally curved shape, sidewall hole 118 can be pointed at the posterior end, flat at one or both ends, or otherwise shaped. As described with reference to FIG. 2, however, a tapered shape can help better distribute impact forces. Regarding the potential for migration of bone cells through side wall holes 118, the migration may include osteoblasts, osteocytes, lining cells, etc.

FIG. 6 is a diagrammatic representation of a cross sectional view of an embodiment of implant 100. FIG. 6 illustrates cavity 102, posterior portion 104, anterior portion 106, medial-lateral side wall 108, and medial-lateral side wall 110. Side wall hole 118 is also illustrated. Medial-lateral edges 124 and 126 also appear in FIG. 6. Fluoroscopy markers 120 and 122 also appear in FIG. 6. Instrument engagement hole 112 and instrument engagement notch 114 are illustrated in FIG. 6. FIG. 6 also shows hole depth dh.

Posterior portion 104 may define instrument engagement hole 112 and adjoining instrument engagement notch 114 as illustrated in FIG. 6. Instrument engagement hole 112 may include threads to engage corresponding features of instrument 12 whereby implant 100 can be inserted in to implantation space 18 of FIG. 1. Instrument engagement notch 114 may engage corresponding features on instrument 12 thereby preventing rotation of implant 100 relative to instrument 12 when instrument 12 and implant 100 are engaged.

FIG. 6 also shows that instrument engagement hole 112 can have a hole depth dh. In some embodiments, hole depth dh can vary between different implants 100 and different sizes of implants 100 or hole depth dh can be the same for all implants 100 in some embodiments. In some embodiments, hole depth dh is 0.17″ for all sizes of implants 100. The dimensions of instrument engagement notch 114 can vary between implants 100, or sizes, or can be the same for all implants 100. In one embodiment, instrument engagement notch 114 can be 0.09″ deep. With posterior portion length Ip (discussed in more detail with reference to FIG. 5) of 0.22″ posterior portion 104 (including a 0.09″ deep notch and 0.17″ deep instrument engagement hole 112) is sufficiently strong to allow instrument 12 (of FIG. 1) to drive implant 100 into implantation space 18. It should be understood, however, that the dimensions provided are for a particular embodiment and that other dimensions can be used.

FIG. 7 is a diagrammatic representation of an oblique view of an embodiment of implant 100 illustrating various features of implant 100. FIG. 7 illustrates cavity 102, posterior portion 104, anterior portion 106, medial-lateral side wall 108, and medial-lateral side wall 110. Side wall hole 118 and regions 117 and 119 are also illustrated. Medial-lateral edges 124, 126, and 128 and corners 134, 136, 138, and 140 also appear in FIG. 7. In various embodiments, posterior corners 134, 136, 138, and 140 can be rounded (See FIG. 6). Cephalad surface 113, caudal surface 115, and teeth 116 are also illustrated by FIG. 7. Fluoroscopy markers 120 also appear in FIG. 7. Instrument engagement hole 112 and instrument engagement notch 114 are illustrated in FIG. 7.

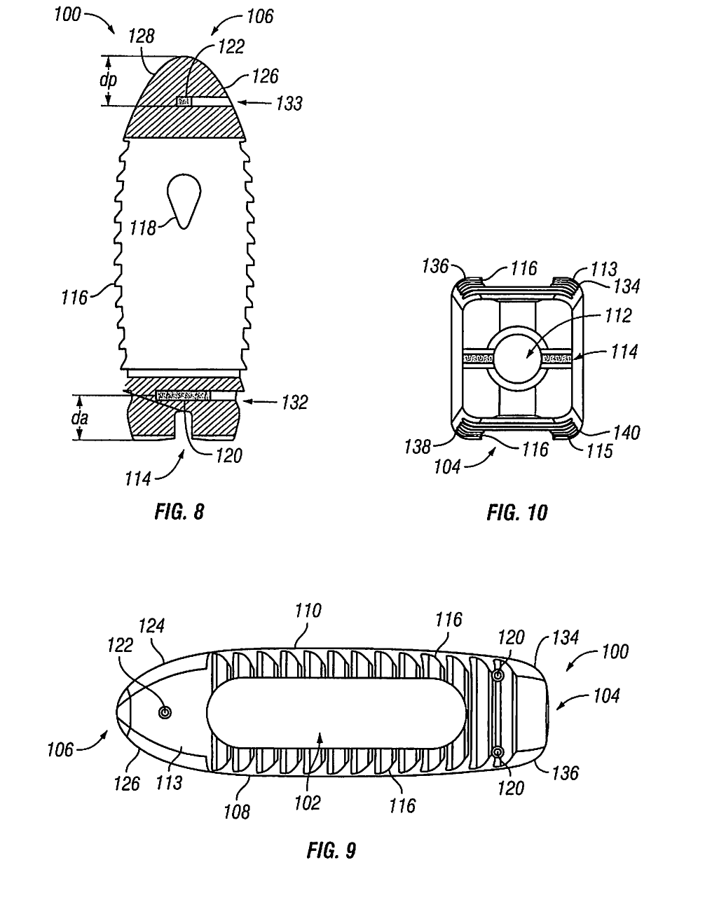

FIG. 8 is a diagrammatic representation of a cross sectional view of an implant 100 according to one embodiment. FIG. 8 illustrates posterior portion 104, anterior portion 106, and side wall hole 118. Medial-lateral edges 126 and 128 also appear in FIG. 8. Fluoroscopy marker 120 and 122 also appear in FIG. 8. Holes 132 and 133 and distances dp and da associated with fluoroscopy markers 120 and 122 are illustrated also. Instrument engagement notch 114 is also illustrated in FIG. 8.

As shown by FIG. 8, fluoroscopy markers 120 can be elongated rods, bars, etc. with fluoroscopy markers 122 being a shorter rod, bar, a bead, ball, or other shape that is easily distinguishable from fluoroscopy markers 120 in at least one viewing plane. For example, fluoroscopy marker 122 can be a shorter rod than fluoroscopy marker 120. Such differences and positioning of fluoroscopy markers 120 and 122 can allow surgical personnel to triangulate the position and orientation of implant 100 in the patient's body. In embodiments where lengths Ip and Ia of posterior and anterior portions 104 and 106 of implant 100 are fixed, fluoroscopy markers 120 and 122 allow surgical personnel to readily determine where implant 100 rests in the patient's body.

Fluoroscopy markers 120 and 122 may be press fit into corresponding holes 132 and 133 on implant 100. Holes 132 and 133 can be dimensioned such that one or more of fluoroscopy markers 120 and 122 are centered about axis of impact 20 (of FIG. 2) although holes 132 and 133 may be dimensioned so that fluoroscopy markers 120 and 122 reside closer to one of cephalad or caudal surfaces 113 or 115 than another.

FIG. 9 is a diagrammatic representation of a top view of an embodiment of implant 100. FIG. 9 illustrates cavity 102, posterior portion 104, anterior portion 106, medial-lateral side wall 108, and medial-lateral side wall 110. Medial-lateral edges 124 and 126 and corners 134 and 136 also appear in FIG. 9. Cephalad surface 113 and teeth 116 are also illustrated by FIG. 9. Fluoroscopy marker 120 and 122 also appear in FIG. 9.

Forces exerted on implant 100 by vertebral bodies (and other sources) can tend to cause implant 100 to move in a posterior direction after it is inserted in the implantation space. To prevent such movement and expulsion of implant 100 from the implantation space, teeth 116 may be formed in cephalad surface 113 (by, in some embodiments, forming grooves in implant 100). By the term “forming teeth in implant 100” it is meant that the teeth do not extend beyond the general contours of cephalad surface 113. This arrangement contrasts with forming teeth on the body of implant 100 by which teeth extend beyond the general contours of cephalad surfaces 113. Forming teeth 116 in the body of implant 100 allows more accurate matching of implant 100 height to implantation space height h then forming teeth 116 on the body of implant 100. Forming teeth 116 in the body of implant 100 also minimizes spinal subsidence after implant 100 is placed in implantation space 18. Implant 100 with teeth formed in the body of implant 100 therefore provides improved recovery and shorter recovery time for the patient. Teeth 116 may be formed into cephalad surface 113 and caudal surface 115. In some embodiments, teeth 116 can be formed by machining, laser, casting, powder metallurgy, etc. In some embodiments, teeth 116 may be added to cephalad surface 113 or otherwise added thereto. Teeth 116 may extend across all, or a portion of, cephalad surface 113. In some embodiments, teeth 116 may lie primarily in the region of cephalad surface 113 near cavity 102 although teeth 116 can extend along cephalad surface 113 beyond the region near cavity 102. Teeth 116 may, in various embodiments, be rounded or sharp, point toward posterior or anterior portions 104 and 106 or a combination thereof.

FIG. 10 is a diagrammatic representation of an end view of one embodiment of implant 100. FIG. 10 illustrates posterior portion 104, corners 134, 136, 138, and 140, cephalad surface 113, caudal surface 115, and teeth 116. Instrument engagement hole 112 and instrument engagement notch 114 are also illustrated in FIG. 10. Implants 100 may include instrument engagement features other than threaded holes such as instrument engagement hole 112. In various embodiments, the instrument engagement features may include mechanisms employing hooks, ball-detents, couplers, etc.

Side wall hole 118 can be elongated in a direction parallel to the axis of impact 20. Side wall hole 118 can be tear drop shaped with the tapered portion of the tear drop shape pointing toward posterior portion 104 in some embodiments. Regardless of the shape of side wall hole 118, side wall hole 118 can be located at about the center of medial-lateral side walls 108 or 110 or can be located elsewhere on medial-lateral side walls 108 and 110. Side wall hole 118 can be positioned such that (when viewed from the side) the center of side wall hole 118 aligns with axis of impact 20 as shown in FIG. 5. Regardless of the shape, orientation, location, or number of side wall holes 118, side wall holes 118 can have the same dimensions for all size of implants 100. In some embodiments, the dimensions of side wall hole 118 can be different for different implants 100 or different sizes of implants 100.

Referring again to FIG. 2, when instrument 12 is impacted to-drive implant 10 in to implantation space 18 (of FIG. 1) stresses are induced in implant 10. The inventors have found, using finite element stress analysis, that the presence of circular side wall hole 418 (see FIG. 4) causes the stress in the medial-lateral side walls 108 or 110 (in which side wall hole 418 is located) to concentrate in regions 417 and 419 above and below side wall hole 418 respectively. The inventors have found that tear drop shaped side wall hole 118 distributes the impaction stresses nearly uniformly in regions 117 and 119.

As used herein, the terms “comprises,” “comprising,” “includes,” “including,” “has,” “having” or any other variation thereof, are intended to cover a non-exclusive inclusion. For example, a process, process, article, or apparatus that comprises a list of elements is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in one embodiment”.

Although embodiments have been described in detail herein, it should be understood that the description is by way of example only and is not to be construed in a limiting sense. It is to be further understood, therefore, that numerous changes in the details of the embodiments and additional embodiments will be apparent, and may be made by, persons of ordinary skill in the art having reference to this description. It is contemplated that all such changes and additional embodiments are within scope of the claims below.

What is claimed is:

1. An interbody spinal fusion implant for insertion between vertebral bodies of a human spine, the vertebral bodies having an anterior aspect and a posterior aspect and a depth therebetween, the implant comprising:
a posterior portion of the implant shaped to interface with an insertion tool;
an anterior portion of the implant opposite the posterior portion of the implant;
a pair of medial-lateral side walls of the implant between the posterior and anterior portions of the implant, wherein the posterior portion, anterior portion and medial lateral sidewalls define an implant body having a cephalad surface, a caudal surface and defining a fusion promotion cavity extending between the cephalad surface and the caudal surface; and
wherein at least one of the side walls defines a hole from the exterior of the implant to the fusion promotion cavity, the side wall hole being substantially centered about a primary axis of impact and being tapered toward the posterior portion of the Implant along the axis of impact.

2. The implant of claim 1, wherein the side wall hole is tear drop shaped.

3. The implant of claim 1, wherein at least one of the surfaces is convex.

4. The implant of claim 1, further comprising at least one of the cephalad or caudal surfaces defining teeth therein and extending beyond the cavity toward at least one of the ends of the implant.

5. The implant of claim 1, wherein the anterior portion of the implant comprises a nose tapered in the saggital and transverse planes.

6. The implant of claim 1, wherein the posterior portion of the implant defines a threaded instrument engagement hole for engagement with an instrument for inserting the implant in an implantation space.

7. The implant of claim 3, wherein the posterior portion of the implant defines an instrument engagement notch adjoining the instrument engagement hole.

8. The implant of claim 1, further comprising a first and a second elongated fluoroscopy marker toward the posterior end of the implant and a third fluoroscopy marker toward the anterior end of the implant.

9. The implant of claim 8, wherein the third fluoroscopy marker has approximately the same cross sectional area as the first fluoroscopy marker in a first viewing plane and has a different cross sectional area than the first fluoroscopy marker in a second viewing plane orthogonal to the first viewing plane.

10. An interbody spinal fusion implant for insertion between vertebral bodies of a human spine, the vertebral bodies having an anterior aspect and a posterior aspect and a depth therebetween, the implant comprising:
a posterior portion of the implant defining a threaded instrument engagement hole for engagement with an instrument for inserting the implant in the implantation space, the posterior portion of the implant defining an instrument engagement notch adjoining the instrument engagement hole, the instrument engagement hole having a depth in a direction substantially along the axis of impact;
an anterior portion of the implant opposite the posterior portion of the implant and including a nose tapered in the saggital and transverse planes;
a first and a second elongated fluoroscopy marker located toward a posterior end of the implant;
a third fluoroscopy marker located toward an anterior end of the implant, wherein the third fluoroscopy marker has approximately the same cross sectional area as the first fluoroscopy marker in a first viewing plane and has a different cross sectional area than the first fluoroscopy marker in a second viewing plane orthogonal to the first viewing plane;
a pair of medial-lateral side walls of the implant between the posterior portion and anterior portion of the implant, wherein the anterior portion, posterior portion, and medial-lateral sidewalls define an implant body having a convex cephalad surface and a convex caudal surface, the implant body defining a fusion promotion cavity extending from the convex cephalad surface to the convex caudal surface;
wherein at least one of the medial-lateral side walls defines a hole from the exterior of the implant to the fusion promotion cavity, the side wall hole being substantially centered about the axis of impact and being tapered towards the posterior portion of the implant; and
wherein at least one of the convex cephalad surface or the convex caudal surface define teeth extending beyond the cavity toward at least one of the anterior portion or posterior portion of the implant, wherein the height of the teeth is defined by a radius of curvature for a corresponding convex surface.

11. The implant of claim 10, wherein the side wall hole is tear drop shaped.

12. The implant of claim 10, wherein the side wall hole is about centered between the posterior and anterior ends of the side wall.

13. A surgical kit for surgery on human spines, the kit comprising:
a set of different sized interbody spinal fusion implants for insertion within implantation spaces formed within a disc space, each implant comprising:
a posterior portion shaped to interface with an insertion tool;
an anterior portion opposite the posterior portion of the implant;
a pair of medial-lateral side walls of the implant between the posterior and anterior portions of the implant, wherein the posterior portion, anterior portion and medial lateral sidewalls define an implant body having a cephalad surface, a caudal surface and defining a fusion promotion cavity extending between the cephalad surface and the caudal surface; and
wherein at least one of the side walls defines a hole from the exterior of the implant to the fusion promotion cavity, the side wall hole being substantially centered about a primary axis of impact and being tapered toward the posterior portion of the implant along the axis of implant.

14. The kit of claim 13, wherein the side wall hole is tear drop shaped.

15. The kit of claim 13, wherein the posterior portion of each implant defines a threaded instrument engagement hole for engagement with the instrument, the posterior portion of each implant defines an instrument engagement notch adjoining the instrument engagement hole, and the instrument engagement hole of each implant has a depth in a direction along the axis of impact, the depth being the same for all sizes.

16. The kit of claim 13, wherein the cephalad and caudal surfaces of each implant are convex, each of the cephalad surfaces having a first radius of curvature, each of the caudal surfaces having a second radius of curvature, the first radius of curvature being the same for all sizes, the second radius of curvature being the same for all sizes.

17. The kit of claim 13, wherein for each implant the posterior portion has a first length from a posterior end to the fusion promotion cavity, the first length being the same for all sizes.

18. The kit of claim 17, wherein for each implant, the anterior portion has a second length from an anterior end to the fusion promotion cavity, the second length being the same for all sizes.

19. The kit of claim 13 wherein each implant further comprises a first and a second elongated fluoroscopy marker toward a posterior end of the implant and a third fluoroscopy marker toward an anterior end of the implant, at least one of the fluoroscopy markers being a distance from one of the ends of the implant, the distance being the same for all sizes.

20. A method of manufacturing an interbody spinal fusion implant for insertion within an implantation space formed across the height of a disc space between vertebral bodies of a human spine, the vertebral bodies having an anterior aspect and a posterior aspect and a depth therebetween, the method comprising:
forming a posterior portion of the implant shaped to interface with an insertion tool;
forming an anterior portion of the implant opposite the posterior end of the implant;
forming a pair of medial-lateral side walls of the implant between the posterior and anterior portions of the implant, wherein the posterior portion, anterior portion and medial lateral sidewalls define an implant body having a cephalad surface, a caudal surface and defining a fusion promotion cavity extending between the cephalad surface and the caudal surface;
forming a hole in at least one of the medial-lateral side walls from the exterior of the implant to the fusion promotion cavity, the side wall hole being substantially centered about a primary axis of impact and being tapered toward the posterior portion of the implant along the axis of impact; and
forming a set of teeth into at least one of the cephalad or caudal surfaces.

21. The method of claim 20, wherein the side wall hole is tear drop shaped.

22. The method of claim 20, further comprising forming the set of teeth to extend beyond the cavity toward at least one of the ends of the implant.

23. The method of claim 20, wherein at least one of the surfaces is convex.

24. The method of claim 20, wherein the cephalad surface is convex and has a radius of curvature and wherein the method further comprises forming the set of teeth into the cephalad surface wherein the height of teeth in the set of teeth does not extend beyond the radius of curvature.

25. The method of claim 20, wherein the caudal surface is convex and has a radius of curvature and wherein the method further comprises forming the set of teeth into the caudal surface wherein the height of teeth in the set of teeth does not extend beyond the radius of curvature.

26. The method of claim 20, further comprising tapering the anterior portion in the saggital and transverse planes.

27. The method of claim 20, wherein the posterior portion of the implant defines a threaded instrument engagement hole for engagement with an instrument for inserting the implant in an implantation space.

28. The method of claim 25, wherein the posterior portion of the implant defines an instrument engagement notch adjoining the instrument engagement hole.

29. The method of claim 20, further comprising:
forming at least a first and second marker hole in the implant toward the posterior end of the implant and a third marker hole toward the anterior end of the implant; and
inserting a first marker in the first fluoroscopy marker in the first marker hole, a second fluoroscopy marker in the second marker hole and a third fluoroscopy marker in the third marker hole.

30. The method of claim 28, wherein the third fluoroscopy marker has approximately the same cross sectional area as the first fluoroscopy marker in a first viewing plane and has a different cross sectional area than the first fluoroscopy marker in a second viewing plane orthogonal to the first viewing plane.
